(19) United States
(12) Patent Application Publication
(10) Pub. No.: US 2012/0015004 A1
(43) Pub. Date: Jan. 19, 2012

(54) ENCAPSULATED SALTS AND USE IN HIGH ACID BEVERAGES

(75) Inventors: Maxim A. Mironov, Ural (RU); Peter S. Given, Ridgefield, CT (US); Teodoro Rivera, Algonquin, IL (US)

(73) Assignee: TROPICANA PRODUCTS, INC., Bradenton, FL (US)

(21) Appl. No.: 13/160,260

(22) Filed: Jun. 14, 2011

Related U.S. Application Data
(60) Provisional application No. 61/355,313, filed on Jun. 16, 2010.

(51) Int. Cl. A61K 9/48; A61K 33/14; B82Y 5/00

(52) U.S. Cl. 424/400; 424/663; 977/773; 977/915

(57) ABSTRACT

Encapsulated nutrient salts including nutrient salt particles encapsulated with a water-insoluble chitosan-stearic acid complex are provided. A method for forming encapsulated nutrient salts is provided, including forming a water-in-oil micro-emulsion including an oil and an aqueous salt solution, adding chitosan and stearic acid to the water-in-oil micro-emulsion, where the chitosan and stearic acid form a complex, and collapsing the aqueous phase of the water-in-oil micro-emulsion to form the encapsulated salt particles.

### CROSS-REFERENCE TO RELATED APPLICATIONS

This application claims priority to U.S. Provisional Application Serial No. 61/355,313, filed on Jun. 16, 2010, which is incorporated herein in its entirety.

### FIELD OF THE INVENTION

The present invention relates to the field of delivering particular ingredients to a consumer in an aqueous system, more particularly encapsulated nutrients such as salts of metals in an aqueous system such as, for example, orange juice.

### BACKGROUND OF THE INVENTION

Consumers demonstrate continued interest in ready-to-drink (RTD) beverages fortified with FDA approved water-soluble nutrients believed to provide health benefits. For example, important nutrients include metal salts such as potassium salts. For example, a potassium intake of at least 4.7 grams a day helps reduce the risk of stroke, hypertension, osteoporosis and kidney stones. Low potassium may contribute to muscle spasms and ‘restless leg syndrome’. Low potassium levels may also contribute to general feelings of fatigue and muscle tiredness. Because potassium is an important part of synthesizing protein and metabolizing glucose and glycogen, prime energy sources for the body, low potassium levels can leave you feeling tired, achy and generally fatigued. Low potassium levels may also exacerbate irritability and anxiety. Studies show low potassium levels are linked with bone loss in osteoporosis.

Generally, many individuals do not regularly consume sufficient amounts of potassium or other nutrient salts. Thus, it would be beneficial to provide potassium or other nutrient salts via a beverage that is consumed regularly by the average person.

However, manufacturing such beverages represents a formidable challenge. As a rule, water-soluble nutrients impact the color and taste of the beverages and/or negatively react with other components of the beverage which affects the way the product is processed, its stability, or its shelf life. This makes simple addition of these nutrients to existing formulations impossible. It would also be desirable to provide potassium or other nutrient salts in a stable form for use in an aqueous system, such as beverages, so that the ingredients can withstand certain process conditions related to mixing, homogenizing and pasteurizing of the beverage, yet would be available as a nutrient once the beverage is consumed by an individual.

### BRIEF SUMMARY OF THE INVENTION

In a first aspect, the invention is directed to encapsulated nutrient salts comprising nutrient salt particles encapsulated with a water-insoluble chitosan-stearic acid complex.

In a second aspect, the invention is directed to a method of forming encapsulated nutrient salts comprising forming a water-in-oil micro-emulsion comprising an oil and an aqueous salt solution; adding chitosan and stearic acid to the water-in-oil micro-emulsion, wherein the chitosan and stearic acid form a complex; and collapsing the aqueous phase of the water-in-oil micro-emulsion to form the encapsulated salt particles.

In a third aspect, the invention is directed to a beverage comprising encapsulated nutrient salts encapsulated with a chitosan-stearic acid complex.

In a fourth aspect, the invention is directed to method of delivering a nutrient salt, comprising encapsulating a nutrient salt with a complex of chitosan and stearic acid; mixing the encapsulated nutrient salt with a beverage; wherein the beverage is to be ingested by a person; and further wherein the encapsulated nutrient salt breaks down upon ingestion allowing the nutrient salt to be released and utilized by the person

### BRIEF DESCRIPTION OF THE DRAWINGS

FIG. 1 depicts the formation of a micro-emulsion with an oil and salt (A) in water.

FIG. 2 depicts the formation of a chitosan-stearic acid complex (B) surrounding the salt (A) in water.

FIG. 3 depicts the collapse of the aqueous phase and formation of micro-particles of encapsulated salt.

### DETAILED DESCRIPTION OF THE INVENTION

The invention is generally directed to delivery systems for water-soluble nutrients, in particular water-soluble salts wherein the salts are present in a beverage in a form that is inert during manufacture and storage, yet completely bioavailable upon consumption. The delivery system allows significant loading of the salt component, in particular potassium. By encapsulating the salts, the salts may be added to high acid beverages with minimal or no flavor off taste or effects on chemistry such as alteration of the pH.

Aspects of the invention are directed to encapsulated nutrient salts comprising salt particles encapsulated with a water-insoluble complex containing a high molecular weight cation and a fatty acid. The nutrient salts may be any suitable nutrient salts such as, but not limited to potassium, sodium, magnesium, calcium, manganese, zinc, selenium salts. Suitable anions include, but are not limited to, chloride, sulfate, carbonate, and phosphate. In particular, the salts are sodium chloride or potassium chloride.

The salt particles are encapsulated with a water-insoluble complex such as a complex of chitosan and stearic acid. Additional aspects relate to a method of encapsulating the salts.

The encapsulated salt particles have a particle diameter size ranging from about 10 nanometers to about 200 microns. The particle size should be small enough not to increase the viscosity of the beverage. The encapsulated salt particles preferably remain encapsulated even in acid solutions having a pH between 2.5 and 5. The system is storage stable for at least 12 months in powder form or in beverages stored at refrigerated or ambient conditions.

In a particular aspect of the invention, nano- or micro-particles (crystals) of salt are encapsulated with a film of chitosan-stearic acid complex. Chitosan-stearic acid complex is a white powder without pronounced taste. This non-soluble polymer film prevents the resolution of the salt in an aqueous media of beverage such as acidic media in a range of pH from 2.5 to 4.3. At the same time this polymer complex is destroyed or disassembled by acidic media and fermentation systems in the stomach and gastrointestinal tract.

A water-in-oil micro-emulsion is formed with a non-polar high-boiling point oil, such as liquid paraffin, vegetable oils, or medium chain triglyceride oils, and an aqueous solution of the nutrient salt. The chitosan-stearic acid complex is then formed from chitosan (cation), stearic acid (fatty acid), and lecithin (surfactant). Salt Particles are then formed via collapse of an aqueous phase in reverse micelles or micro-emulsions. Depending on the size of the salt particles, a colloidal solution or fine suspension is formed. Then the surface of the encapsulated particles may be modified.

Chitosan is a product of chitin modification and produced in large scale from marine crab and shrimp.

Stearic acid is a fatty acid comprising 18 carbons. Other suitable fatty acids could include most saturated fatty acids (for oxidative stability), ranging from C6 to C24, such as fatty acids ranging from C14-C22. The fatty acid may be saturated or unsaturated.

Non-limiting examples of suitable surfactants include propylene glycol alginate, monoglyceride, diglyceride, dioctyl sulfosuccinate sodium (DOSS), polyoxyethylene (20) sorbitan monolaurate (also known as polysorbate 20, available under the trade name Tween® 20 from ICI Americas, Inc. of Wilmington, Delaware), polyoxyethylene (20) sorbitan monopalmitate (also known as polysorbate 40, available under the trade name Tween® 40 from ICI Americas, Inc.), polyoxyethylene (20) sorbitan monostearate (also known as polysorbate 60, available under the trade name Tween® 60 from ICI Americas, Inc.), polyoxyethylene (20) sorbitan tristearate (also known as polysorbate 65, available under the trade name Tween® 65 from ICI Americas, Inc.), polyoxyethylene (20) sorbitan monooleate (also known as polysorbate 80, available under the trade name Tween® 80 from ICI Americas, Inc.), sorbitan monolaurate (available under the trade name Span® 20 from ICI Americas, Inc.), sorbitan monopalmitate (available under the trade name Span® 40 from ICI Americas, Inc.), betaine, sucrose esters of fatty acids, sucrose monomyristate, sucrose palmitate, sucrose stearate, mono and diglycerides of fatty acids, monoglyceride monooleate, monoglyceride monolaurate, monoglyceride monopalmitate, lecithin, diglyceride mixtures, citric acid esters of mono and diglycerides of fatty acids, acetic acid esters of mono and diglycerides of fatty acids, lactic acid esters of mono and diglycerides of fatty acids, mono and diacetyl tartaric esters of mono and diglycerides of fatty acids, polyglycerol esters of fatty acids, cyclodextrins (α, β, or γ), propylene glycol esters of fatty acids, stearoyl lactylates, C₈₋₁₈ free fatty acids, other emulsifiers as is known to those skilled in the art, and combinations thereof. Also included would be saponins, lecithin, phospholipids, lysophospholipids, acacia gum, modified starch, modified acacia gum, beet pectin, and bile acids (e.g., cholic acid).

All of the components are food approved compounds. Except liquid paraffin, no organic solvents and no chemical reagents are used.

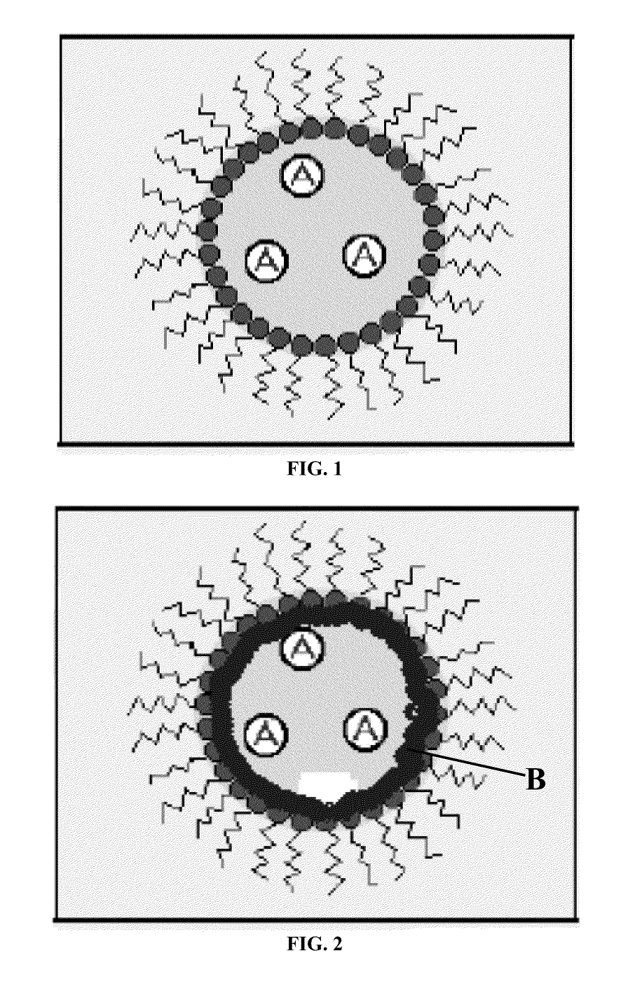

FIG. 1 shows the formation of micro-emulsion. Salt particles are depicted by A. The micro-emulsion can be obtained by vigorous agitation with addition of surfactant such as soybean lecithin. The surfactant regulates the particle sizes. For instance, mechanical micro-emulsion (without surfactant) does not overcome the micron scale. If the size of particles is not a critical parameter, the addition of a surfactant can be avoided.

FIG. 2 shows the formation of a chitosan-stearic acid complex. The chitosan-stearic acid complex is formed in a micro-emulsion by adding pure stearic acid and chitosan to the water-in-oil micro-emulsion formed above.

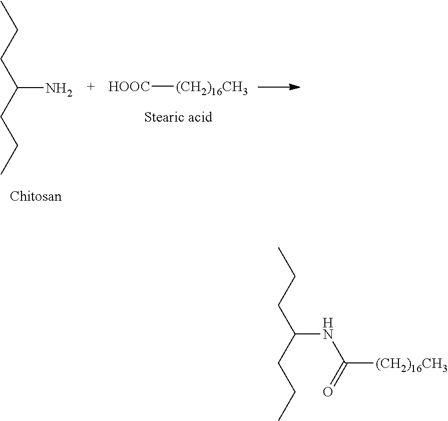

This complex is not a surfactant itself, but the complex has a very high affinity to phase boundary. Therefore, as shown in FIG. 2, the complex migrates to the oil-water boundary (B) during mechanical agitation and forms stable micro- or nano-encapsulated particles. The structure of this complex and methods for its obtaining is described in the following article: Biomacromolecules, 2005, 6, 2416.

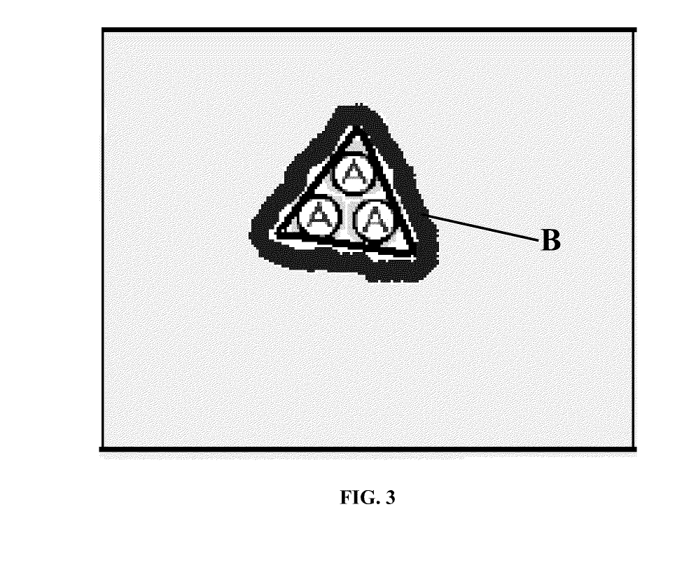

FIG. 3 shows the collapse of the aqueous phase and formation of nano- or micro-particles of salt. The temperature of the aqueous phase is increased to 120-130° C. which leads to evaporation of water and collapse of the aqueous phase. The salt (A) is crystallized from the solution and forms agglomerates encapsulated by the polymeric chitosan-stearic acid complex (B). Due to the high affinity to phase boundary this complex migrates to the new solid surface of the particles and covers defects. As a result, a suspension of salt particles coated with chitosan-stearic acid complex is obtained.

The surface of the complex may be modified by increasing the temperature up to 180° C. to form amide bonds between amino groups of chitosan and the carboxyl of the stearic acid. This modification makes the surface very stable towards diluted acid and aqueous media of beverages. Other possible transformations include cross-linkage with pectin (Polymer Bulletin, 2005, 55, 367), hydrophilization of surface with levulinic acid, and many others.

In a particular aspect, the salt particles are encapsulated in accordance with the following steps:

1) Agitating a concentrated aqueous salt solution and a non-polar, high-boiling point oil, to form a micro-emulsion. (Optionally, a surfactant is also included in the solution.) This step may include mechanical particle size reduction such as high shear mixing, homogenization, or microfluidization.

2) Adding the polymer cation and fatty acid and further agitating the micro-emulsion.

3) Heating the micro-emulsion to about 120-130° C. for a time sufficient to collapse the aqueous phase and crystallize the encapsulated salt to form a suspension of small encapsulated salt particles.

4) Heating the suspension to at least 160° C. and up to about 250° C., for example 175 to 180° C., to modify the surface of the encapsulated particles.

5) Filtering and washing the encapsulated salt particles.

In step 1, the concentration of the salt in the aqueous solution may be up to 50%, typically between 20 and 30%, and in one aspect 25%. The salts may be any suitable salts such as, but not limited to potassium, sodium, magnesium, calcium, manganese, zinc, selenium salts. Suitable anions include, but are not limited to, chloride (Cl), sulfate (SO₄), carbonate (CO₃), and phosphate (PO₄). Particular examples include, but are not limited to, potassium chloride or sodium chloride. The amount of liquid paraffin is an amount sufficient form the desired water-in-oil micro-emulsion, generally 5-95%, typically 10-50%. The ratio of oil to aqueous “salt” phase is 0.5:1 to 10:1, typically 2:1 to 7:1.

In step 2, the polymer cation is chitosan and the fatty acid is stearic acid. Generally, but not limited to, about 0.5 to 10 wt % chitosan is combined with about 0.1 to 20 wt % stearic acid. The agitation is for a time suitable to form the chitosan-fatty acid complex.

In step 3, the time sufficient to collapse the aqueous phase and crystallize encapsulated salt is generally the time it takes to drive off all of the water vapor, generally about 45 to 120 minutes,

In step 4, the stability of the encapsulated surface can be varied via formation of very strong amide bonds between stearic acid and chitosan under thermal conditions. Generally, this step takes up to 3 hours depending on the extent of the surface modification. This approach is amenable for large scale production of encapsulated salt components.

In step 5, the suspension of particles may be diluted with a solvent such as hexane, petroleum ether, alcohol, or supercritical carbon dioxide (essentially any solvent that will dissolve and wash-off/remove the oil phase), filtered off, and then washed with the solvent. As a final stage, the microparticles may be modified with pectin and levulinic acid. Such modifications may make a thicker surface layer to prevent salt from migrating into the RTD beverage, or to provide a net negative surface charge which helps keep separate particles from joining together to form very large particles in the RTD beverage over shelf life.

The process of preparing the chitosan-stearic acid complex may use any suitable equipment. For example, a turbine or other effective emulsifying mixer may be used to mix the ingredients. A filtrator or other filtration device may be used to filter the encapsulated salt product. A suitable dryer may be used to provide high temperatures (e.g., up to 200° C.).

As noted, the stearic acid-chitosan salt complex is formed via reverse micelles or a water-in-oil micro-emulsion. The aqueous phase contains a concentrated solution of salt, for example 25% potassium chloride. A viscous non-polar oil with a high boiling point provides the micro-emulsion. Suitable non-polar oils may by liquid paraffin or mineral oil, vegetable oils, or medium-chain triglyceride oils. Vegetable oils may be saturated or unsaturated. Saturated vegetable oils in the C14 to C20 range are suitable. Example vegetable oils include, but are not limited to sunflower, safflower (and high oleic versions of both), canola oil, rapeseed oil, corn oil, olive oil, palm oil, palm kernal oil, coconut oil, cocoa butter, shea oil, chia seed oil, cranberry seed oil, flax seed oil, fish oil, and algal oils.

Further aspects of the invention relate to the use of the encapsulated nutrient salts in liquid beverages. The liquid beverage can be orange juice. The orange juice can either be not-from-concentrate (“NFC”) or from-concentrate (“FC”) juice. The beverage can also be other types of citrus or non-citrus juices, for example, 100% juices (e.g., apple and grape) and 1% to 90% juice cocktails (e.g., cranberry and grapefruit). Other beverages include, for example, dairy drinks, energy drinks, sports drinks, fortified/enhanced water drinks, soy drinks, fermented drinks (e.g., yogurt and kefir), carbonated drinks, hybrid mixtures of juice and dairy drinks, including both bottle and can products and fountain syrup applications.

Importantly, the encapsulated salts of the present invention are able to withstand not only the rigorous processing methods as disclosed herein, but are able to be broken down when ingested. Encapsulated functional ingredients used in the invention can be achieved using enzymes in the human body or a number of other mechanisms, such temperature or duration. For example, it is preferable that encapsulants are stomach soluble (or soluble in gastric acid).

The encapsulation matrix will preferably be broken down in the stomach or the gastrointestinal tract to expose the nutrient salt. Once broken down within the human body, the salts are available to be utilized by the body in such helpful ways as intended. As described above, each salt has a positive healthy effect upon ingestion.

In addition, it is contemplated that the encapsulated salt within a beverage can include various additional ingredients, such as flavoring agents, sweeteners, coloring agents, stabilizers and pH adjusters, as desired for the particular use. Other additives are also contemplated. The encapsulated salts can be added to the beverage either pre or post pasteurization.

Various amounts of the encapsulated salts can be incorporated into a beverage to provide a desired amount of the encapsulated salts per serving of the beverage. The amount may vary depending on the application and nutritional content desired. For example, in orange juice, functional ingredients may be added in an amount between about 5 to 7000 mg of encapsulated salts per 8 fluid ounces (0.24 liters)(serving size). The amount of encapsulated salts also may be varied to account for taste, mouthfeel, visual appearance, shelf-life, efficacy levels approved, qualified health claims and other such characteristics and considerations. Other amounts are also contemplated within the scope of the invention as would be appreciated by those of ordinary skill in the art.

The encapsulated salts are sufficiently mixed in the beverages to provide a relatively uniform distribution; however, mixing is not limited to dissolving the functional ingredients in a liquid. For example, the functional ingredients may be mixed in powder form with a powdered drink mix (e.g., Gatorade® or other sports beverages) to form a substantially evenly blended powdered product. The salts may be spray dried on a carrier (e.g., maltodextrin) for ease of dissolution, etc. or fluidized bed dried on a carrier. It is also possible to add the encapsulated salts via a complementary package method such as a cap or a pre-packaged straw.

It is also contemplated that the beverages may include functional ingredients other than the encapsulated salts. The beverages may also include other nutritional or non-nutritional ingredients other than the functional ingredient. Vitamins, minerals or combinations thereof may be added to the beverages. Ingredients such as flavorings, sweeteners, colorings, thickeners, stabilizers, emulsifiers, pH adjusters, acidulants, electrolytes, proteins, carbohydrates and preservatives also can be added. Other additives are also contemplated. The ingredients can be added at various points during processing, including prior to pasteurization, with or without the encapsulated functional ingredient, and after pasteurization.

The finished food beverages with the encapsulated functional ingredient may have a shelf life of about 6-12 months and possibly up to 24 months under ambient conditions, depending on the level of processing the beverage undergoes, the type of packaging and the material used for packaging the beverage and the conditions of storage. Additional factors that may affect the shelf-life of the beverage include, for example, the nature of the base formula (e.g., a beverage sweetened with sugar has a longer shelf-life than a beverage sweetened with aspartame) and environmental conditions (e.g., exposure to high temperatures and sunlight is deleterious to ready to drink (RTD) beverages)).

In addition, it is contemplated that encapsulated salts according to aspects of the present invention will not affect desired physical properties. For example, it is contemplated that encapsulated salts will not affect acceptable mouthfeel, or physical and chemical interactions with the mouth, or affect the taste of the finished product.

Mixing should be accomplished such that the encapsulated salt is not destroyed. The mixer(s) can be selected for a specific application based, at least in part, on the type and amount of ingredients used, amount of ingredients used, the amount of product to be produced and the flow rate. Generally, a commercially available mixer, such as those available from Invensys APV of Getzville, N.Y. or Silverson Machines, Inc. of East Longmeadow, Mass., may be used.

The beverages may be homogenized and/or pasteurized. Beverages may, in addition be further or post processed following the adding of the encapsulated salts. Post processing can include, for example, cooling the product solution and filling it into container for packaging and shipping. Post processing may also include deaeration of the food product to <4.0 ppm oxygen, preferably <2.0 ppm and more preferably <1.0 ppm oxygen. Deaeration, however, and other post processing tasks may be carried out prior to processing, prior to pasteurization, prior to mixing with the encapsulated salt and/or at the same time as adding the encapsulated salt. In addition, an inert gas (e.g., nitrogen) headspace may be maintained during the intermediary processing of the product and final packaging. Additionally/alternatively, an oxygen barrier and/or oxygen scavengers could be used in the final packaging.

### Example 1

A homogeneous micro-emulsion was obtained from 10 ml of liquid paraffin and 2 ml of 25% solution of potassium chloride in water. An effective laboratory high shear mixer was used. In some experiments soybean lecithin (100/200/300 mg) was added to the micro-emulsion, as it helps reduce the particle size. Subsequently, 450 mg of stearic acid and 300 mg of fine-dispersed chitosan were added to the micro-emulsion. The reaction mixture was intensively agitated for 20 min, and during this time a suspension of microparticles was formed. Then the temperature was increased up to 120-130° C. for 1 hour and then up to 200° C. for 3 hours. The resulting suspension was diluted with hexane (20-30 ml), filtered off, and washed with hexane to remove the oil phase. As a final stage, microparticles could be modified with pectin and levulinic acid. The product is a white powder (around 1 g depending on conditions) without any pronounced taste.

The mechanism of release included disintegration of amide bond between stearic acid and chitosan under digestive fermentation systems, coupled with swelling of chitosan in acidic media of stomach. This combined action releases salt for availability and absorption in the gastrointestinal tract.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

We claim:

1. Encapsulated nutrient salts comprising nutrient salt particles encapsulated with a water-insoluble chitosan-fatty acid complex.

2. The encapsulated nutrient salts of claim 1 wherein the nutrient salt particles are selected from potassium, sodium, magnesium, calcium, manganese, zinc, or selenium salts of anions selected from chloride, sulfate, carbonate, or phosphate.

3. The encapsulated nutrient salts of claim 1 wherein the nutrient salt particles are selected from the group consisting of potassium chloride and sodium chloride.

4. The encapsulated nutrient salts of claim 1 wherein the nutrient salt particles are potassium chloride.

5. The encapsulated nutrient salts of claim 1 wherein the nutrient salt particles have a particle diameter size ranging from about 10 nanometers to about 100 microns.

6. The encapsulated nutrient salts of claim 1 wherein the encapsulated nutrient salt particles remain encapsulated in acid solutions having a pH between 2.5 and 4.3.

7. The encapsulated nutrient salts of claim 1 wherein the polymer complex is destroyed or disassembled by acidic media and fermentation systems in the stomach and gastrointestinal tract.

8. A method of forming encapsulated nutrient salts comprising
a. forming a water-in-oil micro-emulsion comprising an oil and an aqueous salt solution;
b. adding chitosan and stearic acid to the water-in-oil micro-emulsion, wherein the chitosan and stearic acid form a complex; and
c. collapsing the aqueous phase of the water-in-oil micro-emulsion to form the encapsulated salt particles.

9. The method of claim 8 wherein the oil is selected from the group consisting of liquid paraffin, vegetable oils, and medium chain triglyceride oils.

10. The method of claim 8 wherein the salts are selected from potassium, sodium, magnesium, calcium, manganese, zinc, or selenium salts of anions selected from chloride, sulfate, carbonate, or phosphate.

11. The method of claim 8 wherein the salts are selected from the group consisting of potassium chloride and sodium chloride.

12. The method of claim 8 wherein the salts are potassium chloride.

13. The method of claim 8 wherein in step b, the micro-emulsion is heated to about 120-130° C. for a time sufficient to collapse the aqueous phase and crystallize the encapsulated salt to form a suspension of the encapsulated salt particles.

14. The method of claim 13 further comprising modifying the surface of the encapsulated salt particles by heating the suspension up to 200° C.

15. A beverage comprising encapsulated nutrient salts encapsulated with a chitosan-stearic acid complex.

16. The beverage of claim 15 wherein the salts are selected from potassium, sodium, magnesium, calcium, manganese, zinc, or selenium salts of anions selected from chloride, sulfate, carbonate, or phosphate.

17. The beverage of claim 15 wherein the salt particles are potassium chloride or potassium chloride.

18. The beverage of claim 15 wherein the encapsulated salt particles have a particle diameter size ranging from about 10 nanometers to about 100 microns.

19. The beverage of claim 15 wherein the beverage has a pH of between 2.5 and 4.3 and the encapsulated salt particles remain encapsulated in the beverage.

20. The beverage of claim 15 wherein the polymer complex is destroyed or disassembled by acidic media and fermentation systems in the stomach and gastrointestinal tract upon consumption of the beverage.

21. The beverage of claim 15 wherein the beverage is selected from juice or a sport drink.

22. A method of delivering a nutrient salt, comprising the steps of:
encapsulating a nutrient salt with a complex of chitosan and stearic acid;
mixing the encapsulated nutrient salt with a beverage; wherein the beverage is to be ingested by a person; and
further wherein the encapsulated nutrient salt breaks down upon ingestion allowing the nutrient salt to be released and utilized by the person.

23. The method of claim 22 wherein the beverage is selected from juice or a sport drink.
